(19) United States
(12) Patent Application Publication
(10) Pub. No.: US 2020/0000756 A1
(43) Pub. Date: Jan. 2, 2020

(54) NEOSTIGMINE COMBINATION AND COMPOSITIONS

(71) Applicant: GT BIOPHARMA, INC., Washington, DC (US)

(72) Inventor: Kathleen CLARENCE-SMITH, Washington, DC (US)

(73) Assignee: GT BioPharma, Inc., Washington, DC (US)

(21) Appl. No.: 16/480,177

(22) PCT Filed: Jan. 23, 2018
(86) PCT No.: PCT/US2018/014901

§ 371 Date: Jul. 23, 2019

Related U.S. Application Data
(60) Provisional application No. 62/449,699, filed on Jan. 24, 2017.

(51) Int. Cl. A61K 31/196; A61K 9/00; A61K 31/4178; A61P 21/04

(52) U.S. Cl. CPC A61K 31/196; A61K 9/0014; A61P 21/04; A61K 31/4178; A61K 9/0053

(57) ABSTRACT

The present invention describes the use of a 5HT3-antagonist, in combination with neostigmine, to facilitate the treatment of a patient suffering from myasthenia gravis or other myasthenic syndromes by providing a therapeutically effective neostigmine bromide or methylsulfate daily dose that attenuates or even abrogates the dose-limiting gastrointestinal adverse effects of neostigmine.

### RELATED APPLICATIONS

This application claims the benefit of U.S. Provisional Patent Application Ser. No. 62/449699, filed Jan. 24, 2017, the disclosure of which is incorporated herein in its entirety by reference.

### FIELD OF THE INVENTION

This invention pertains to the field of the treatment of the symptoms of muscle weakness associated with myasthenia gravis (MG) and other myasthenic syndromes in mammalian subjects, particularly including humans, dogs, and cats suffering from these diseases.

### OBJECT OF THE INVENTION

The present invention provides a new composition and method to enable the safe administration of neostigmine to mammalian subjects with myasthenic syndromes, including MG, with said composition comprising combinations, including fixed-dose combinations, of an antagonist of the 5-hydroxytryptamine subtype-3 receptor (“5HT3-antagonist”) with an effective dose of neostigmine.

### BACKGROUND OF THE INVENTION

Myasthenia gravis (MG) is a chronic autoimmune disease of the neuromuscular junction (NMJ) caused by antibodies that attack components of the postsynaptic membrane, impair neuromuscular transmission, and lead to varying degrees of weakness and fatigue of skeletal muscle. The prevalence of MG in the United States is estimated at 14 to 20 per 100,000 population, with approximately 36,000 to 60,000 cases in the United States (Howard, 2015). However, MG remains underdiagnosed and the prevalence is probably higher. The disease has also been described in dogs, and cats (Shelton, 2016).

The hallmark of the disease is muscle weakness that increases during periods of activity and improves after periods of rest. Muscular weakness can be generalized or localized to certain muscle groups, and involvement of the bulbar and respiratory muscles can be life threatening (Phillips and Vincent, 2016). Groups of muscles are often involved in typical patterns. Certain muscles such as those that control eye and eyelid movement, facial expression, chewing, talking, and swallowing are often, but not always, involved in the disorder. The muscles that control breathing and neck and limb movements may also be affected.

MG occurs in all ethnic groups and both genders. It most commonly affects young adult women (under 40) and older men (over 60), but it can occur at any age (Myasthenia Gravis Fact Sheet; National Institute of neurological Disorders and Stroke, 2016). In neonatal myasthenia, the fetus may acquire immune proteins (antibodies) from a mother affected with MG. Generally, cases of neonatal MG are temporary and the child's symptoms usually disappear within 2-3 months after birth (Myasthenia Gravis Fact Sheet; National Institute of neurological Disorders and Stroke, 2016). Other children develop MG indistinguishable from occurrences in adults. MG in juveniles is uncommon (Myasthenia Gravis Fact Sheet; National Institute of neurological Disorders and Stroke, 2016).

The basic abnormality in MG is a reduction in nicotinic acetylcholine receptors (AChRs) at neuromuscular junctions due to the effects of autoantibodies that are directed against the AChRs in most patients, or against neighboring proteins involved in the clustering of AChRs, such as MuSK, LRP-4, or agrin (Drachman, 2016).

The diagnosis may be missed during the early stages of the disease, and depends on the recognition of clinical manifestations, the measurement of autoantibodies, and/or electrophysiological features (Drachman, 2016).

Rarely, children may show signs of congenital myasthenia or congenital myasthenic syndrome (CMS). These are not autoimmune disorders, but are caused by defective genes that produce abnormal proteins instead of those that normally are involved in cholinergic transmission: acetylcholinesterase (the enzyme that breaks down acetylcholine), acetylcholine receptors, and other proteins present along the muscle membrane (Engel, 2012).

In some rare cases, a myasthenic syndrome is due to bi-allelic variants in the gene encoding the vesicular acetylcholine transporter (VAChT) located in the presynaptic terminal (O'Grady et al, 2016). In other cases, degeneration of the nerves that innervate muscles such as occurs with aging (Lexel, 1997) leads to a myasthenic syndrome. Recently (Makarious et al, 2017), have reported on a myasthenic syndrome involving an emerging toxicity of checkpoint inhibitors used for the treatment of certain malignancies. Most individuals with CMS, or with an immune-oncology therapy-related myasthenic syndrome, or with progressive age-related degeneration of the motor neurons that innervate muscles, benefit from the same treatment as those that are effective in patients with autoimmune MG, namely choline esterase (ChE) inhibitors (Engel 2012; Abicht et al, 2003 updated in 2014).

Ocular myasthenia gravis (OMG) is a localized form of myasthenia gravis in which autoantibodies directed against acetylcholine receptors block or destroy these receptors at the postsynaptic neuromuscular junction. The hallmark of OMG is a history of painless weakness or fatigability of the extraocular muscles and ptosis with normal pupillary function and visual acuity. Clinical, laboratory, electrophysiologic, and pharmacologic tests are available for diagnosis. Treatment can begin with symptom management; there is no cure (Smith and Lee, 2017).

The treatment of myasthenic syndromes involves treatment of the symptoms through the enhancement of cholinergic transmission at the neuromuscular junction by acetylcholine esterase inhibitors (AChEIs) that do not appreciably cross the Blood-Brain-Barrier (BBB), such as neostigmine. Patients with autoimmune-related myasthenic syndromes may also benefit from immunotherapy to slow disease progression. Options for immunosuppression include corticosteroids, azathioprine, mycophenolate mofetil, cyclosporine, tacrolimus, methotrexate, rituximab, cyclophosphamide, intravenous immunoglobulin, plasmapheresis, and thymectomy (Gotterer and Li, 2016).

Neostigmine treats the symptoms by retarding the enzymatic hydrolysis of acetylcholine at cholinergic synapses, so that acetylcholine concentrations increase at the neuromuscular junction and the effect of acetylcholine is both increased and prolonged. ChE inhibitors have been shown to cause considerable improvement in some patients and little to none in others (Howard, 2015). Strength rarely returns to normal. Neostigmine bromide (Prostigmin), iodide or methylsulfate, all of which do not appreciably cross the BBB, are commonly used for the treatment of MG. No fixed dosage schedule suits all patients.

Neostigmine is commercially available as a brand or generic drug, for example as oral Prostigmin®, consisting of tablets comprising 15 mg neostigmine bromide and vials for parenteral injection comprising 0.5 mg of neostigmine methylsulfate, a 15 mg of neostigmine bromide oral dose being equivalent to 0.5 mg of neostigmine methylsulfate parenteral dose.

A neostigmine bromide slow release preparation which can be taken once every day for treating myasthenia gravis is described in CN 102258492, the contents of which are incorporated herein in their entirety by reference.

Neostigmine is also described in combination with some plant extracts according to traditional Chinese medicine (CN 102552381), for treating myasthenia gravis.

A process for the synthesis of neostigmine iodide and neostigmine methylsulfate is disclosed in RU 2010130899, the disclosure of which is incorporated herein in its entirety by reference.

Neostigmine methylsulfate has been disclosed as a remedy for eye diseases, in an eye drop preparation, consisting of a neostigmine methylsulfate aqueous solution, also containing other chemicals, emulsified with an oily higher fatty acid solution obtained from olive oil and isopropyl myristate (JPS 56104814, the contents of which are incorporated herein in their entirety by reference).

Neostigmine methylsulfate has also been disclosed, in combination with naphazoline hydrochloride and chlorpheniramine maleate, for the treatment of conjunctivitis (CN 105708838, the disclosure of which is incorporated herein in its entirety by reference).

The need for neostigmine varies from day-to-day and during the same day in response to infection, menstruation, emotional stress, and hot weather. Gastro-intestinal adverse effects of neostigmine used to treat MG are dose-limiting and typically consist of gastrointestinal complaints, queasiness, loose stools, nausea, vomiting, abdominal cramps, and diarrhea (Howard, 2015).

Gastro-intestinal side effects are an important source of discomfort for the patient, may be a source of non-compliance, or may result in the need to decrease the daily dose of neostigmine to mitigate these side effects whereupon these side effects become dose-limiting. As a consequence, efficacy is reduced.

Normally, said gastro-intestinal side effects, in particular when using neostigmine methylsulfate intravenous injection (0.5 mg/ml vials or 1 mg/ml in 10-ml multiple dose vials), are counteracted by a previous or concurrent administration of glycopyrrolate ad recommended in the label for injectable neostigmine (Bloxyberz

Prescribing Information, revised May 2013).

However, the literature does not disclose how to safely treating MG with neostigmine without the undesired gastro-intestinal dose-limiting adverse effects that are inevitably associated to said treatment. Thus, the problem of providing safe, chronic treatment of MG and other myasthenic syndromes with neostigmine therapeutic or even at higher maximally effective doses remains unsolved.

### DEFINITIONS

“MG”: Myasthenia Gravis. MG is a chronic neuromuscular autoimmune disease, characterized by muscle weakness. The basic abnormality in MG is a reduction in the number or function of acetylcholine nicotinic receptors (AChRs) at neuromuscular junctions due to the effects of autoantibodies. About 85% of patients with generalized MG have antibodies to AChRs. Antibodies to other proteins at the neuromuscular junction are present in some cases of MG, such as antibodies to muscle-specific kinase, or to low density lipo-protein 4, or to agrin.
“Myasthenic syndrome”: refers to conditions associated with muscle weakness in which the cholinergic transmission at the neuromuscular junction is decreased either because of a decrease in the number and/or dysfunction of post-synaptic nicotinic receptors or to a decrease in the amount of acetylcholine (“Ach”) available at the neuromuscular junction due to gene mutations in the presynaptic proteins involved in the synthesis, storage and release of ACh, or to degeneration of cholinergic nerves that innervate muscles. An emerging myasthenic syndrome (with or without auto antibodies to nicotinic receptors) has been reported in association with immune-therapies used for the treatment of certain malignancies. Myasthenic syndromes are sometimes loosely referred to as MG in the medical literature but herein, all MG-like conditions which do not involve autoantibodies to nicotinic receptors will be referred to as myasthenic syndromes. MG itself is a myasthenic syndrome and is considered as such herein, although, as the most prominent myasthenic syndrome it is often mentioned specifically (as in the phrase “MG and other myasthenic syndromes”).
“Effective dose of 5HT3-antagonist” this expression, as used herein, refers to a single dose of said 5HT3-antagonist that is at least as high as the dose preventing or treating nausea and vomiting in a mammalian subject. Said single dose is from 1 to 300 mg, normally from 0.01 mg/kg to 1.8 mg/kg of body weight.
“Effective daily dose of 5HT3-antagonist”: this expression, as used herein, refers to a daily dose of said 5HT3-antagonist that is at least as high as the dose preventing or treating nausea and vomiting in pediatric or adult human patients undergoing cancer chemotherapy, said effective daily dose being from 0.03 mg/kg to 3 mg/kg of body weight.
“Neostigmine”: unless otherwise specified, this term, as used herein, refers to a pharmaceutically acceptable salt of neostigmine (“neostigmine pharmaceutically acceptable salt”), the daily doses and the amounts per unit form thereof being expressed as equivalents of neostigmine bromide per oral unit forms, and equivalents of neostigmine methylsulfate per injectable unit forms.
“Effective daily dose of neostigmine”: this expression, as used herein, refers to a neostigmine pharmaceutically acceptable salt daily dose, including doses used in the titration period, equivalent to at least 15 mg of neostigmine bromide administered orally or to at least 0.5 mg of neostigmine methylsulfate administered parenterally.
“Maximally effective (daily) dose” or “Maximal effective (daily) dose”, as used herein for neostigmine, refers to any neostigmine daily dose allowing the expression of significantly greater neostigmine efficacy, heretofore hindered by the typical gastro-intestinal neostigmine adverse effects.
“Effective amount per unit form”, referring to neostigmine, is a neostigmine amount per unit form equivalent to at least 0.2 mg of neostigmine methylsulfate in a parenteral 1 ml-solution unit form or as released from a transdermal drug delivery system; or, respectively, a neostigmine amount per unit form equivalent to at least 15 mg of neostigmine bromide in an oral unit form.
“Neostigmine bromide” or “neostigmine methyl sulfate”: these expressions, or equivalent ones, as used herein in connection with neostigmine doses, refer to a neostigmine dose per unit form or to a neostigmine daily dose (range) equivalent of either neostigmine bromide, in the case of an oral dose, or to neostigmine methylsulfate, in the case of a parenteral dose.
“Mammal” or “mammalian subject” as used herein refer to any class of warm-blooded higher vertebrates (such as placentals, marsupials, or monotremes) that nourish their young with milk secreted by mammary glands, have the skin usually more or less covered with hair; and include, but are not limited to, a human, a dog, and a cat.

### SUMMARY OF THE INVENTION

It has now been found that, by using a 5-HT3 receptor antagonist, also referred to as 5-HT3 receptor inhibitor or simply 5HT3-antagonist, in constant combination with neostigmine, it is possible to treat symptoms of muscle weakness associated with MG and other myasthenic syndrome in mammalian subjects, and particularly humans, dogs, and cats, suffering from myasthenia gravis and other myasthenic syndromes by maintaining a therapeutically effective neostigmine bromide daily dose or a therapeutically effective neostigmine methylsulfate daily dose with little to no dose-limiting gastro-intestinal adverse effect.

In particular, the constant combination of a 5HT3-antagonist with neostigmine enables for the first time greater or complete efficacy of neostigmine in the treatment of symptoms of muscle weakness associated with MG and other myasthenic syndromes.

Thus, the present invention provides a method for treating symptoms of muscle weakness associated with MG and other myasthenic syndromes, which comprises administering to a mammalian subject in need of said treatment a combination of a 5HT3-antagonist with an effective daily dose of neostigmine.

Any of the 5HT3-antagonists disclosed in the literature may be used in combination with a dose of neostigmine that is generally at least as high as that currently used for treating myasthenia gravis. The chronic use of this combination improves the symptoms of myasthenia gravis by concurrently mitigating or even eliminating the gastro-intestinal dose-limiting adverse effects induced by neostigmine, thus enabling the safe administration of the recommended or even higher than recommended dose of neostigmine (maximally effective dose), leading to greater efficacy and safety of neostigmine.

According to the present invention, preferably, the 5HT3-antagonists used are those shown to be effective for preventing or treating nausea and vomiting following cancer chemotherapy. In fact, surprisingly, 5-HT3 receptor inhibitors, known to block nausea, vomiting, and diarrhea induced by chemotherapeutic drugs, have been shown, in particular when administered at high doses, to also block the gastro-intestinal side effects of neostigmine without affecting its efficacy in treating symptoms of muscle weakness associated with MG or other myasthenic syndromes, thus allowing the administration of neostigmine maximally effective doses.

This finding is surprising also because, notwithstanding the gravity of the illness and the fact that both neostigmine and the 5HT3-antagonists were two families of products in use during more than a decade, each in its own indication, to date nobody thought that, by combining an effective dose of 5HT3-antagonist with an effective dose of neostigmine, it would have been possible to safely improve the conditions of patients suffering from MG and other myasthenic syndromes.

In addition, this combination has the triple advantage of (1) allowing for an increase of the currently used neostigmine doses, thus attaining greater neostigmine efficacy; (2) allowing for the parenteral administration of neostigmine at least up to certain doses, without the need of pretreatment with glycopyrrolate, as recommended for example, in the neostigmine methylsulfate package inserts; and (3) allowing for the continuous chronic infusion of neostigmine methylsulfate while minimizing or even abrogating gastro-intestinal side effects, thus enabling maximally effective doses to be administered.

Thus, the present invention provides a method for treating symptoms of muscle weakness associated with MG and other myasthenic syndromes, which comprises administering to mammalian subjects, and in particular, humans, dogs, and cats, in need of said treatment an effective daily dose of a 5HT3-antagonist in combination with an effective daily dose of a pharmaceutically acceptable salt of neostigmine.

According to an embodiment, the invention provides a pharmaceutical combination comprising a 5HT3-antagonist, at a daily dose that is at least as high as the pediatric or adult dose shown to be effective for the prevention or treatment of chemotherapy-induced nausea and vomiting, and a maximally effective daily dose of a neostigmine pharmaceutically acceptable salt.

According to another embodiment, the invention provides a 5HT3-antagonist, in a pharmaceutical composition comprising, as an active ingredient, said 5HT3-antagonist in an amount at least as high as the pediatric or adult dose shown to be effective for the prevention or treatment of chemotherapy-induced nausea and vomiting, in admixture with a pharmaceutical carrier, for use for preventing or attenuating the dose-limiting gastrointestinal adverse effects of neostigmine in the treatment of symptoms of muscle weakness associated with MG and other myasthenic syndromes in a mammalian subject in need of said treatment.

According to a further embodiment, the invention includes the use of a 5HT3-antagonist for the preparation of a medicament including a pharmaceutical composition comprising, as an active ingredient, said 5HT3-antagonist, in an amount per unit form at least as high as the pediatric or adult dose shown to be effective for the prevention or treatment of chemotherapy-induced nausea and vomiting (effective amount per unit form), in admixture with a pharmaceutical carrier, for preventing, attenuating or even abrogating the gastrointestinal adverse effects of neostigmine in the treatment of symptoms of muscle weakness associated with MG and other myasthenic syndromes, in a mammalian subject in need of said treatment.

As set forth above, the amount per unit form of the 5HT3-antagonist is at least as high as the pediatric or adult dose shown to be effective for the prevention or treatment of chemotherapy-induced nausea and vomiting and may be up to 4 times said dose.

Said composition, comprising said 5HT3-antagonist, for the first time allows the administration of maximally effective neostigmine doses to mammalian subjects suffering from symptoms of muscle weakness associated with MG or other myasthenic syndromes, with the consequent expression of the neostigmine greater efficacy.

According to yet a further embodiment, the invention provides a pharmaceutical fixed-dose combination including a pharmaceutical composition in dosage unit form comprising a 5HT3-antagonist, in an amount per unit form that is at least as high as the pediatric or adult dose shown to be effective for the prevention and treatment of chemotherapy-induced nausea and vomiting, as Component (a) and an effective amount per unit form of a neostigmine pharmaceutically acceptable salt, as Component (b), in admixture with a pharmaceutical carrier or vehicle.

According to a preferred embodiment, the invention provides a pharmaceutical combination comprising an approved 5HT3-antagonist, at a dose that is at least as high as the pediatric or adult dose approved for the prevention or treatment of chemotherapy-induced nausea and vomiting, and an effective, especially maximally effective, dose of a neostigmine pharmaceutically acceptable salt.

According to an aspect of this preferred embodiment, the invention provides an approved 5HT3-antagonist, in a pharmaceutical composition comprising, as an active ingredient, said 5HT3-antagonist in an amount at least as high as the pediatric or adult dose approved for the prevention or treatment of chemotherapy-induced nausea and vomiting, in admixture with a pharmaceutical carrier, for use for preventing, attenuating or even abrogating the gastrointestinal adverse effects of neostigmine in the treatment of myasthenia gravis and other myasthenic syndromes.

According to a further aspect of this preferred embodiment, the invention includes the use of an approved 5HT3-antagonist for the preparation of a medicament consisting of a pharmaceutical composition comprising, as an active ingredient, said 5HT3-antagonist, in an amount at least as high as the pediatric or adult dose approved for the prevention or treatment of chemotherapy-induced nausea and vomiting, in admixture with a pharmaceutical carrier, for preventing, attenuating or even abrogating the gastrointestinal adverse effects of neostigmine in the treatment of myasthenia gravis and other myasthenic syndromes.

As set forth above, the amount of the 5HT3-antagonist is at least as high as the pediatric or adult dose approved for the prevention or treatment of chemotherapy-induced nausea and vomiting and may be up to 4 times said dose.

In the above combination, including fixed-dose combinations, said amount per unit form of said 5HT3-antagonist administered orally Component (a) in said composition normally is from 1 μg to 300 mg.

When the 5HT3-antagonist is ondansetron hydrochloride dihydrate, administered by continuous infusion, the doses per continuous infusion are equivalent to from 0.5 mg/h (12 mg per 24 hours) to 1 mg/h (24 mg/day) of ondansetron base.

In the above combination, including fixed-dose combinations, the neostigmine Component (b) administered orally, in a pharmaceutical composition in dosage unit form, is present in said composition in an amount per unit form of from 15 mg to 200 mg.

In the above combination, including fixed-dose combinations, the neostigmine Component (b) administered parenterally, in a pharmaceutical composition in dosage unit form, is present in said composition in an amount per unit form of from 0.5 mg to 240 mg.

Ondansetron may also be present in a slow-release composition.

The dose of neostigmine, normally as methyl sulfate, administered by intravenous injection, is 0.03 mg/kg to 0.28 mg/kg administered as an intravenous bolus.

According to yet a further aspect of this preferred embodiment, the invention provides a pharmaceutical fixed-dose combination comprising a pharmaceutical composition comprising a 5HT3-antagonist, in an amount per unit form that is at least as high as the pediatric or adult dose approved for the prevention and treatment of chemotherapy-induced nausea and vomiting, as Component (a) and an effective amount per unit form of a neostigmine pharmaceutically acceptable salt, as Component (b), in admixture with a pharmaceutical carrier or vehicle.

The oral dose of neostigmine, normally as bromide, in an IR tablet will preferably range from 1 mg to 200 mg, normally from 15 mg to 75 mg, depending on safety and tolerability. When the 5HT3-antagonist is ondansetron, the dose per tablet in combination with neostigmine will range from 0.5 mg to 32 mg, normally from 4 mg to 16 mg or from 4 mg to 8 mg.

### DETAILED DESCRIPTION

The present invention provides, according to its aspects,

a method for safely improving the conditions or symptoms of muscle weakness of mammalian subjects, and particularly, humans, dogs, and cats, suffering from MG or other myasthenic syndromes by treating said patient with a 5HT3-antagonist in combination with neostigmine;
a 5HT3-antagonist, for use in the treatment of MG and other myasthenic syndromes in combination with neostigmine;
the use of a 5HT3-antagonist for the preparation of a medicament for the treatment of symptoms of muscle weakness associated with MG and other myasthenic syndromes in combination with neostigmine; and
a fixed-dose combination comprising a pharmaceutical composition in dosage unit form comprising, as active ingredients, a 5HT3-antagonist Component (a) and neostigmine Component (b).

### The 5HT3-Antagonist

Any 5HT3-antagonist may be used for allowing the safe treatment of MG and other myasthenic syndromes with normal, but also with high and very high, maximally effective neostigmine doses. Antagonists of the 5HT3 receptor that are shown to be effective for the prevention or treatment of chemotherapy-induced nausea and vomiting are particularly useful according to the present invention.

The 5HT3-antagonist is preferably selected from the group consisting of 5-methyl-2-[(4-methyl-1H-imidazol-5-yl)methyl]-2,3,4,5-tetrahydro-1H-pyrido[4,3-b]indol-1-one (alosetron) and pharmaceutically acceptable salts and solvates thereof, especially its hydrochloride, disclosed in U.S. Pat. No. 5,360,800; (±)-6-chloro,3,4-dihydro-4-methyl-3-oxo-N-(quinuclidinyl)-2H-1,4-benzoxazine-8-carboxamide (azasetron) and pharmaceutically acceptable salts and solvates thereof, especially its hydrochloride, disclosed in U.S. Pat. No. 4,892,872; [(1S,5R)-8-methyl-8-azabicyclo[3.2.1]octan-3-yl]3,5-dichlorobenzoate (bemesetron, CAS: 40796-97-2); (10R)-10-[(2-methyl-1H-imidazol-1-yl)methyl]-5,6,9,10-tetrahydro-4H-pyrido(3,2,1-jk)carbazol-11-one (cilansetron) and pharmaceutically acceptable salts and solvates thereof, especially its hydrochloride monohydrate, disclosed in U.S. Pat. No. 4,939,136; (3R)-10-oxo-8-azatricyclo[5.3.1.0^{3,8}]undec-5-yl 1H-indole-3-carboxylate (dolasetron) and pharmaceutically acceptable salts and solvates thereof, especially its monomethanesulfonate monohydrate, disclosed in U.S. Pat. No. 4,906755; (+)-(R)-8,9-dihydro-10-methyl-7-[(5-methylimidazol-4-yl)methyl]pyrido[1,2-a]indol-6(7H)-one (fabesetron) and pharmaceutically acceptable salts and solvates thereof, especially its hydrochloride or maleate, disclosed in U.S. Pat. No. 5,141,945; 1-methyl-N-((1R,3r,5S)-9-methyl-9-azabicyclo[3.3.1]nonan-3-yl)-1H-indazole-3-carboxamide (granisetron) and pharmaceutically acceptable salts and solvates thereof, especially its hydrochloride, disclosed in U.S. Pat. No. 4,886,808; 2,3-dihydro-N-(8-methyl-8-azabicyclo[3.2.1] oct-3-yl)-2-oxo-1H-benzimidazole-1-carboxamide (itasetron) and pharmaceutically acceptable salts and solvates thereof, especially its hydrochloride, disclosed in U.S. Pat. No. 5,223,511; 1-phenylmethyl-2-(1-piperazinyl)-1H-benzimidazole (lerisetron) and pharmaceutically acceptable salts and solvates thereof, specially its hydrochloride, disclosed in U.S. Pat. No. 5,256,665 and, in a transdermal preparation, in U.S. Pat. No. 6,136,807; 6-fluoro-5-methyl-2-[(5-methyl-1H-imidazol-4-yl)methyl]-2,3,4,5-tetrahydro-1H-pyrido[4,3-b]indol-1-one (lurosetron, CAS 128486-54-4) and pharmaceutically acceptable salts and solvates thereof, especially its mesylate (GR 87442 N); (±) 1,2,3,9-tetrahydro-9-methyl-3-[(2-methyl-1H-imidazol-1-yl)methyl]-4H-carbazol-4-one (ondansetron) and pharmaceutically acceptable salts and solvates thereof, especially its hydrochloride dihydrate, disclosed in U.S. Pat. No. 4,695578; (3a5)-2-[(S)-1-azabicyclo[2.2.2]oct-3 -yl]-2,3,3a,4, 5,6-hexahydro-1-oxo-1H-benz[de]isoquinoline (palonosetron) and pharmaceutically acceptable salts and solvates thereof, especially its hydrochloride, disclosed in U.S. Pat. No. 5,202,333; 1-methylindol-3-yl)-[(5R)-4,5,6,7-tetrahydro-3H-benzimidazol-5-yl]methanone (ramosetron) and pharmaceutically acceptable salts and solvates thereof, especially its fumarate, disclosed in U.S. Pat. No. 5,344,927; endo-N-(8-methyl-8-azabicyclo[3.2.1]oct-3-y1)-2,3-dihydro-3,3-dimethyl-indole-1-carboxamide (3,3-dimethyl-N-1αH, 5αH-tropan-3α-yl-1-indolinecarboxamide, ricasetron, CAS 117086-68-7) and pharmaceutically acceptable salts and solvates thereof, especially its hydrochloride; the (3-endo)-8-methyl-8-azabcyclo[3.2.1]oct-3-yl ester of 1H-indole-3-carboxylic acid (3-tropanylindole-3-carboxylate, tropisetron) and pharmaceutically acceptable salts and solvates thereof, especially its hydrochloride, disclosed in U.S. Pat. No. 4,789,673; and 5-chloro-2,2-dimethyl-N-(8-methyl-8-azabicyclo[3.2.1]oct-3-yl)-2,3 -dihydro-1-benzofuran-7-carboxamide (zatosetron) and pharmaceutically acceptable salts and solvates thereof, especially its maleate, disclosed in U.S. Pat. No. 5,563,148; the disclosures of all the US patents cited in this paragraph being incorporated herein in their entirety by reference.

Illustrative examples of pharmaceutically acceptable salts of said 5HT3-antagonists include addition salts with inorganic or organic acids such as hydrochloric acid, hydrobromic acid, sulfuric acid, sulfamic acid, phosphoric acid, nitric acid, acetic acid, propionic acid, stearic acid, glycolic acid, oxalic acid, succinic acid, lactic acid, maleic acid, hydroxymaleic acid, fumaric acid, malic acid, tartaric acid, citric acid, ascorbic acid, phenylacetic acid, glutamic acid, benzoic acid, salicylic acid, 2-acetoxybenzoic acid, methanesulfonic acid, ethanesulfonic acid, 2-hydroxyethanesulfonic (isethionic) acid, p-toluenesulfonic acid, 2-naphthalenesulfonic acid, 4-amino-benzenesulfonic (sulfanilic) acid,2,6-naphthalenedisulfonic acid,1,5-naphthalenedisulfonic acid, and pamoic (embonic) acid. Said salt may be solvated with a solvent, said solvent normally being water.

Antagonists of the 5-HT3 receptor that are approved for the prevention or treatment of chemotherapy-induced nausea and vomiting are particularly useful according to the present invention. In particular, azasetron hydrochloride, commercially available in 10-mg tablets; dolasetron monomethanesulfonate monohydrate (also referred to as dolasetron mesylate), commercially available in 200-mg maximal dose tablet; granisetron hydrochloride, commercially available in 2.24-mg maximal dose tablet; ondansetron hydrochloride dihydrate, commercially available in 10-mg maximal dose (equivalent to 8 mg ondansetron base) tablets, and in a 2 mg/ml (in ondansetron base) solution available as a 20-ml multidose vial; palonosetron hydrochloride, commercially available in 0.56-mg tablets, and in 0.075 mg/1.5 ml or 0.25 mg/5 ml (in palonosetron base) vials; and tropisetron hydrochloride, commercially available in 5.64-mg capsules and in 2.265 mg-vial (corresponding to 2 mg of tropisetron base); are the preferred 5HT3-antagonists.

For the treatment of symptoms of muscle weakness associated with MG or other myasthenic disorders by oral route, in combination with neostigmine, the 5HT3-antagonist is administered at a single dose of from 0.001 mg/kg to 1.8 mg/kg of body weight, given from one to three times per day, with a maximum of 300 mg/day.

According to the present invention, the 5HT3-antagonist is used in a pharmaceutical or veterinary composition comprising, as an active ingredient, said 5HT3-antagonist in an amount per unit form of from 1 μg to 300 mg, in admixture with a pharmaceutical carrier or vehicle, and is administered at a daily dose of from 1 μg to 300 mg.

Thus, for example, a pharmaceutical composition according to the present invention to be chronically administered in combination with neostigmine may comprise a 5HT3-antagonist selected from the group consisting of azasetron and pharmaceutically acceptable salts and solvates thereof, in an amount per unit form equivalent to from 5 mg to 10 mg of azasetron hydrochloride, to be administered at a daily dose equivalent to from 15 mg to 40 mg of azasetron hydrochloride; dolasetron and pharmaceutically acceptable salts and solvates thereof, in an amount per unit form equivalent to from 25 mg to 200 mg of dolasetron mesylate, to be administered at a daily dose equivalent to from 75 mg to 200 mg of dolasetron mesylate; granisetron and pharmaceutically acceptable salts and solvates thereof, in an amount per unit form equivalent to from 0.5 mg to 2 mg granisetron base, to be administered at a daily dose equivalent to from 1.5 mg to 8 mg of granisetron base; ondansetron and pharmaceutically acceptable salts and solvates thereof, in an amount per unit form equivalent to from 0.5 mg to 16 mg, normally from 2 mg to 8 mg of ondansetron base, to be administered at a daily dose equivalent to from 6 mg to 64 mg, normally from 6 mg to 32 mg of ondansetron base; palonosetron and pharmaceutically acceptable salts and solvates thereof, in an amount per unit form equivalent to from 0.25 mg to 0.5 mg of palonosetron base, to be administered at a daily dose equivalent to from 0.75 to 2 mg of palonosetron base; and tropisetron and pharmaceutically acceptable salts and solvates thereof, in an amount per unit form equivalent to from 2.5 mg to 5 mg of tropisetron base, to be administered at a daily dose equivalent to from 7.5 mg to 20 mg of tropisetron base.

Preferably, said 5HT3-antagonist is selected from the group consisting of azasetron hydrochloride, in an amount per unit form equivalent to from 5 mg to 10 mg to be administered at a daily dose equivalent to from 15 mg to 40 mg of azasetron hydrochloride; dolasetron mesylate, in an amount per unit form equivalent to from 25 mg to 200 mg of dolasetron mesylate, to be administered at a daily dose equivalent to from 75 mg to 200 mg; granisetron hydrochloride, in an amount per unit form equivalent to from 0.5 mg to 2 mg granisetron base, to be administered at a daily dose equivalent to from 1.5 mg to 16 mg, normally of from 2 mg to 8 mg; ondansetron hydrochloride dihydrate, in an amount equivalent to from 0.5 mg to 32 mg, normally from 2 mg to 32 mg, from 2 mg to 16 mg or from 2 mg to 8 mg ondansetron base, to be administered at a daily dose equivalent to from 6 mg to 64 mg, normally from 6 to 32 mg of ondansetron base; palonosetron hydrochloride, in an amount equivalent to from 0.25 mg to 0.5 mg palonosetron base, to be administered at a daily dose equivalent to from 0.75 to 2 mg of palonosetron base; and tropisetron hydrochloride, in an amount equivalent to from 2.5 mg to 5 mg tropisetron base, to be administered at a daily dose equivalent to from 7.5 to 20 mg of tropisetron base.

In the case of pediatric or obese patients, or also in the case of mammals such as cats and dogs, the daily dose may be decided on the basis of the body weight. Thus, for example, in combination with neostigmine, azasetron hydrochloride may be administered at a daily dose (in kg of body weight) of 0.4-0.5 mg/kg, dolasetron mesylate may be administered at a daily dose of 1.8 mg/kg, up to a maximum dose of 100 mg, normally of 9-9.5 mg/kg, granisetron hydrochloride may be administered at a daily dose of 0.09-0.11 mg/kg, ondansetron hydrochloride dihydrate may be administered at a daily dose of 0.45-0.55 mg/kg, palonosetron hydrochloride may be administered at a daily dose of 0.03 mg/kg and tropisetron hydrochloride may be administered at a daily dose of 0.5-0.6 mg/kg.

More particularly, in pediatric patients the normal single ondansetron hydrochloride dihydrate oral doses (in ondansetron base and in kg of body weight) are from 0.3 mg to 0.5 mg/kg, given every three hours, for neonates and infants, 0.9 mg/kg for a 3-kg baby; of 4 mg for a 8-15 kg child; from 6 mg to 8 mg for a 15-30 kg child; and the same as for adults and children weighing more than 30 kg.

The above doses are preferably administered by oral route.

According to the present invention, in the case of treatment of a patient with intravenous bolus of neostigmine methylsulfate (normally from 0.03 mg/kg to 0.28 mg/kg), or a continuous subcutaneous or transdermal infusion of neostigmine methylsulfate (0.16 mg/kg/day to 4 mg/kg/day), said treatment may be made in combination with a parenteral (intravenous or continuous subcutaneous) injection of a 5HT3-antagonist selected from the group consisting of palonosetron hydrochloride, preferably at a dose, in palonosetron, of 0.25 mg or 0.5 mg, ramosetron hydrochloride, preferably at a dose, in ramosetron, of 3 mg, and ondansetron hydrochloride dihydrate, at a dose, in ondansetron, of from 0.5 mg to 32 mg, normally from 2 mg to 24 mg or from 8 mg to 24 mg, preferably from 8 mg to 16 mg.

When the 5HT3-antagonist is ondansetron hydrochloride dihydrate, administered by continuous infusion, the doses per continuous infusion are equivalent to from.0.021 mg/h to 1.34 mg/h, preferably from 0.5 mg/h to 1 mg/h of ondansetron base.

According to another aspect of the present invention, the pharmaceutical composition comprising a 5HT3-antagonist may contain another active ingredient, in particular a pharmaceutically acceptable salt of neostigmine, co-formulated with said 5HT3-antagonist, in admixture with a pharmaceutical carrier.

### The Neostigmine

Neostigmine is currently indicated for the oral treatment of MG, as neostigmine bromide, in particular in 15-mg tablets for IR administration; and, as parenteral treatment for the reversal of the effects of non-depolarizing neuromuscular blocking agents (NMBAs) after surgery as neostigmine methylsulfate, in 0.5 mg/ml and 1 mg/ml in 10 ml multiple-dose vials.

According to the FDA approved label for oral neostigmine for the treatment of MG, in order to have a more complete response to said treatment, neostigmine bromide oral doses up to 375 mg/day should be administered. However, as set forth above, said doses are not tolerated in most patients.

Higher neostigmine doses than the currently recommended doses should provide further improvement and even a near-to-complete response, i.e., the complete alleviation of symptoms.

According to the present invention, by constantly combining (with a concurrent administration) neostigmine bromide or neostigmine methylsulfate with a 5HT3-antagonist, said treatment becomes safe, and greatly increased effective oral doses, up to 1500 mg/day, and even more, or parenteral doses up to 240 mg/day, and even higher, up to 500 mg/day by continuous 24h-infusion, may be attained without appreciable gastrointestinal adverse effects.

In general, in combination with a 5HT3-antagonist, a pharmaceutically acceptable salt of neostigmine is administered at a unit dose equivalent to from 0.03 mg/kg to 6.25 mg/kg of neostigmine bromide or neostigmine methylsulfate. This unit dose includes an oral unit form comprising an amount of said neostigmine equivalent to from 0.2 mg to 200 mg of neostigmine bromide and a parenteral unit form comprising a neostigmine amount equivalent to from 0.09 mg to 500 mg.

It is hereby specified that, in the particular case of the subcutaneous continuous 24-infusion route, the term “unit dose” is intended as both a unit form and daily dose.

In combination with a 5HT3-antagonist, neostigmine is administered to a mammal at a unit dose, including titration doses, equivalent to from 0.25 mg/kg to 2.5 mg/kg of body weight of neostigmine bromide by oral route, or equivalent to from 0.03 mg/kg to 6.25 mg/kg, normally from 0.03 mg/kg to 4 mg/kg of body weight of neostigmine methylsulfate by parenteral route.

In particular the parenterally administered neostigmine unit dose is equivalent to from 0.03 mg/kg to 0.28 mg/kg of neostigmine methylsulfate by intravenous bolus injection and from 0.03 mg to 8.33 mg/kg, normally from 0.2 mg/kg to 4 mg/kg of neostigmine methylsulfate by subcutaneous continuous 24h-infusion.

More particularly,

for the administration by oral route, the neostigmine oral unit dose normally corresponds to an unit form comprising said neostigmine in an amount per unit form equivalent to from 1 mg to 200 mg of neostigmine bromide;

for the administration by subcutaneous, continuous infusion route, neostigmine is in a parenteral unit dose equivalent to from 0.16 mg/24 hours (“mg/24 h”) to 500 mg/24 h of neostigmine methylsulfate; and for the administration by bolus intravenous route, neostigmine is in an unit form (ampoule or vial) comprising a parenteral unit dose corresponding to an unit form comprising a neostigmine amount equivalent to from 0.09 mg to 0.28 mg of neostigmine methyl sulfate.

The amount of neostigmine, normally as bromide, in an oral Immediate Release (“IR”) unit form (“amount per unit form”) will range from 1 mg to 200 mg, normally from 15 mg to 200 mg, advantageously from 17.5 mg to 200 mg, from 35 mg to 200 mg, from 45 mg to 200 mg, from 62.5 mg to 200 mg, from 70 mg to 200 mg, or from 100 mg to 200 mg, depending on safety and tolerability (per day the oral dose is from 15 mg to 1500 mg, and even more, normally from 17.5 mg to 1500 mg, from 17.5 mg to 1125 mg, from 17.5 mg to 750 mg, or from 17.5 mg to 375 mg). One appropriate neostigmine bromide IR-tablet or IR-capsule comprises 3 mg, 8 mg, 15 mg, 17.5 mg, 35 mg, 50 mg, 62.5 mg, 70 mg, 100 mg, or 200 mg of neostigmine bromide.

Thus, the present invention provides appropriate unit forms, normally a pharmaceutical composition in tablets or capsules comprising, as an active ingredient, a pharmaceutically acceptable salt of neostigmine, in an amount per unit form equivalent to from 17.5 mg to 200 mg, from 35 mg to 200 mg, from 50 mg to 200 mg, from 62.5 mg to 200 mg, from 70 mg to 200 mg or from 100 mg to 200 mg of neostigmine bromide, in admixture with a pharmaceutical carrier or vehicle. Said unit forms may be safely administered to a mammalian subject suffering from symptoms of muscle weakness associated with MG and other myasthenic syndromes, constantly and concurrently with a 5HT3-antagonist. Tablets each comprising a neostigmine pharmaceutically acceptable salt in an amount per tablet equivalent to 17.5 mg, 35 mg, 50 mg, 62.5 mg, 70 mg, 100 mg and 200 mg of neostigmine bromide are particularly appropriate.

Said unit forms are given several times per day at given intervals depending on the patient's response. The normal, maximally effective neostigmine oral daily dose is equivalent to 1200 mg/day of neostigmine bromide, but some patients may need more (up to 1500 mg or more) and some may need less.

In particular, such an oral unit form is destined to be administered from two to seven times per day to mammalian subjects, and particularly, humans, dogs, and cats, suffering from conditions or symptoms of muscle weakness associated with MG or other myasthenic syndromes, in combination with a 5HT3-antagonist.

In the case of administration of high doses, two unit forms may be simultaneously administered from two to seven times per day to said mammalian subjects in combination with a 5HT3-antagonist. In this case, the unit dose thus administered does not correspond to an unit form.

For the continuous 24-hour/day subcutaneous neostigmine infusion, the maximally effective daily dose in combination with a 5HT3-antagonist is equivalent to from 0.2 mg (to neonates) daily to 500 mg daily of neostigmine methylsulfate. Said infusion is in unit doses normally corresponding to the 24-hour dose, preferably in unit doses comprising an amount of neostigmine equivalent to from 0.2 mg to 10 mg, from 10 mg to 50 mg, from 50 mg to 100 mg. from 100 mg to 150 mg, from 150 mg to 200 mg, from 200 mg to 250 mg, from 250 mg to 300 mg, from 300 mg to 350 mg, from 350 to 400 mg, from 400 mg to 450 mg or from 450 mg to 500 mg of neostigmine methyl sulfate.

When the 5HT3-antagonist is ondansetron hydrochloride dihydrate, the doses for continuous infusion are equivalent to from 0.5 mg/h (12 mg per 24 hours) to 1 mg/h (24 mg/day) of ondansetron base. Neostigmine methylsulfate and ondansetron hydrochloride dihydrate appear compatible with each other in an injectable solution. In addition, two separate neostigmine methylsulfate and ondansetron hydrochloride dihydrate solutions may be concurrently administered by using a dual-chamber pump.

When administered by continuous subcutaneous injection, neostigmine methylsulfate is normally administered at single ampoule doses of from 0.09mg (to neonates) to 500 mg, to be administered once every 24 hours in order to supply a maximally effective daily dose of from 1 mg (neonates) to 500 mg.

A safer administration is assured by combining, in the same oral unit form, a 5HT3-antagonist, in an amount per oral unit form of from 1 μg to 300 mg; and neostigmine, in an amount per unit form equivalent to from 0.2 mg to 200 mg, normally from 15 mg to 200 mg, advantageously from 17.5 mg to 200 mg of neostigmine bromide.

A pharmaceutical composition in dosage unit form comprising neostigmine or a pharmaceutically acceptable salt thereof in an amount equivalent to from 17.5 mg to 200 mg of neostigmine bromide or neostigmine methylsulfate, in admixture with a pharmaceutical carrier or vehicle is novel and represents a further object of the present invention.

Preferably, said 5HT3-antagonist is one of the approved 5HT3-antgonists illustrated in “The 5HT3-antagonist” section, in an amount per unit form as illustrated in the same section and said neostigmine is neostigmine bromide or neostigmine methyl sulfate.

### First Aspects of the Invention

According to a first aspect, the present invention provides a method for safely improving the conditions or symptoms of muscle weakness associated with mammalian subjects, and particularly, humans, dogs, and cats, suffering from MG or other myasthenic syndromes by treating said mammalian subject with a 5HT3-antagonist in combination with neostigmine.

More particularly, the present invention proposes a method to safely improve the conditions of patients suffering from MG or other myasthenic syndromes and treated with neostigmine by chronically administering to said patients a 5HT3-antagonist.

In carrying out the method of the present invention, the daily dose of these 5HT3-antagonists is at least as high as that preventing or treating nausea and vomiting in pediatric or adult patients under cancer chemotherapy according to the current protocols for said treatment. In particular, said daily dose is from 1 μg to 300 mg.

The 5HT3-antagonists allowing the safe treatment of neostigmine, in particular at heretofore intolerable doses and even at high doses, are illustrated in “The 5HT3-antagonist” section.

Preferably, said 5HT3-antagonist is selected from the group consisting of azasetron and pharmaceutically acceptable salts and solvates thereof, especially its hydrochloride, at a daily dose equivalent to from 15 mg to 20 mg of azasetron hydrochloride; dolasetron and pharmaceutically acceptable salts and solvates thereof, especially its mesylate monohydrate, at a daily dose equivalent to from 75 mg to 200 mg of dolasetron mesylate; granisetron and pharmaceutically acceptable salts and solvates thereof, especially its hydrochloride, at a daily dose equivalent to from 1.5 mg to 8 mg of granisetron base; ondansetron and pharmaceutically acceptable salts and solvates thereof, especially its hydrochloride dihydrate, at a daily dose equivalent to from 6 mg to 32 mg of ondansetron base; palonosetron and pharmaceutically acceptable salts and solvates thereof, especially its hydrochloride, at a daily dose equivalent to from 0.1 mg to 2 mg, preferably from 0.25 mg to 0.5 mg of palonosetron base; ramosetron and pharmaceutically acceptable salts and solvates thereof, especially its hydrochloride, at a daily dose equivalent to from 75 mcg to 100 mcg of ramosetron hydrochloride; and tropisetron and pharmaceutically acceptable salts and solvates thereof, especially its hydrochloride, at a daily dose equivalent to from 7.5 mg to 20 mg of tropisetron base.

Among the above 5HT3-antagonists to be used in combination, including fixed-dose combinations, with neostigmine, ondansetron and pharmaceutically acceptable salts or solvate thereof, dolasetron and pharmaceutically acceptable salts or solvates thereof, palonosetron and pharmaceutically acceptable salts or solvates thereof, and ramosetron and pharmaceutically acceptable salts or solvates thereof, are particularly advantageous.

The above daily doses of the above 5HT3-antagonists allow the safe administration of high neostigmine daily doses. In particular, the above daily doses of said 5HT3-antagonists allow the safe treatment of adult patients suffering from MG or other myasthenic syndromes with a neostigmine oral daily maximally effective dose equivalent to from 375 mg to 1500 mg, normally from 375 mg to 1200 mg, from 375 mg to 1125 mg, from 375 mg to 750 mg or from 375 mg to 450 mg.

The above daily doses of 5HT3-antagonists also allow the safe administration to a mammalian subject of parenteral doses of neostigmine, normally as methylsulfate. For example, ondansetron hydrochloride dihydrate, at a daily dose equivalent to from 2 mg to 64 mg, normally from 2 mg to 32 mg of ondansetron base allows the safe, continuous 24-hour/day subcutaneous neostigmine infusion, at a greater effective daily dose equivalent to from 10 mg to 500 mg, advantageously from 30 mg to 400 mg, normally from 120 mg to 240 mg of neostigmine methyl sulfate.

### Second Aspect of the Invention

According to a second aspect, the invention provides a 5HT3-antagonist, for use for the safe treatment of mammalian subjects, and particularly, humans, dogs, and cats, suffering from conditions or symptoms of muscle weakness associated with MG or other myasthenic syndromes, in combination with neostigmine. Such a treatment safely improves said conditions or symptoms.

Any 5HT3-antagonist, in particular those that are shown to be effective for the prevention or treatment of chemotherapy-induced nausea and vomiting may be used, in a combination, including a fixed-dose combination, with neostigmine according to this aspect of the present invention. Preferably, said 5HT3-antagonists are those approved for the prevention or treatment of chemotherapy-induced nausea and vomiting.

For said treatment, said 5HT3-antagonist is formulated in a pharmaceutical composition in dosage unit form comprising an effective amount of said 5HT3-antagonist, in admixture with a pharmaceutical carrier or vehicle.

The amounts per unit form of said 5HT3-antagonists and the daily doses to be administered to a patient suffering from MG or other myasthenic syndromes in combination with neostigmine are illustrated in “The 5HT3-antagonist” section.

More particularly said 5HT3-antagonist in said composition is selected from the group consisting of azasetron and pharmaceutically acceptable salts and solvates thereof, in an amount per unit form equivalent to from 5 mg to 10 mg of azasetron hydrochloride, to be administered at a daily dose equivalent to from 15 mg to 40 mg of azasetron hydrochloride; dolasetron and pharmaceutically acceptable salts and solvates thereof, in an amount per unit form equivalent to from 25 mg to 200 mg of dolasetron mesylate, to be administered at a daily dose equivalent to from 75 mg to 200 mg of dolasetron mesylate; granisetron and pharmaceutically acceptable salts and solvates thereof, in an amount per unit form equivalent to from 0.5 mg to 2 mg granisetron base, to be administered at a daily dose equivalent to from 1.5 mg to 8 mg of granisetron base; ondansetron and pharmaceutically acceptable salts and solvates thereof, in an amount per unit form equivalent to from 0.5 mg to 32 mg, normally from 2 mg to 32 mg, from 2 mg to 16 mg or from 2 mg to 8 mg of ondansetron base, to be administered at a daily dose equivalent to from 6 mg to 64 mg, normally from 6 mg to 32 mg of ondansetron base or from 8 mg to 24 mg of ondansetron base; palonosetron and pharmaceutically acceptable salts and solvates thereof, in an amount per unit form equivalent to from 0.25 mg to 0.5 mg of palonosetron base, to be administered at a daily dose equivalent to from 0.75 to 2 mg of palonosetron base; and tropisetron and pharmaceutically acceptable salts and solvates thereof, in an amount per unit form equivalent to from 2.5 mg to 5 mg of tropisetron base, to be administered at a daily dose equivalent to from 7.5 mg to 20 mg of tropisetron base.

Ondansetron and pharmaceutically acceptable salts and solvates thereof, dolasetron and pharmaceutically acceptable salts and solvates thereof, palonosetron and pharmaceutically acceptable salts and solvates thereof and ramosetron and pharmaceutically acceptable salts and solvates thereof are particularly preferred in said composition.

Said composition is for use for safely improving the conditions or symptoms of muscle weakness associated with mammalian subjects, and particularly, humans, dogs, and cats, suffering from MG or other myasthenic syndromes, in combination with a neostigmine pharmaceutically acceptable salt.

Said composition allows a safe treatment of MG or other myasthenic syndromes, in combination with neostigmine daily oral doses equivalent to from 15 mg to 1500 mg, especially of maximally effective daily oral doses of from 375 mg to 1500 mg, normally from 450 mg to 1200 mg, of neostigmine bromide.

Said composition also allows the safe administration to a mammalian subject of parenteral doses of neostigmine, normally as methyl sulfate.

For example, ondansetron hydrochloride dihydrate, by continuous infusion of from 0.5 mg/h (12 mg over 24 hours) to 1 mg/h (24 mg/day), in ondansetron base, allows a safe, continuous 24-hour/day subcutaneous neostigmine methylsulfate infusion, at a maximally effective daily dose of from 50 mg to 500 mg. As set forth above, said concurrent infusion may be made by mixing ondansetron hydrochloride dihydrate and neostigmine methylsulfate in the same vial or by separate, concomitant infusion of the two solutions from two separate dispensers, via a dual-chamber pump.

### Third Aspect of the Invention

According to a third aspect, the invention provides the use of a 5HT3-antagonist for the preparation of a medicament for the treatment of conditions or symptoms of muscle weakness associated with of mammalian subjects, and particularly, humans, dogs, and cats, suffering from MG or other myasthenic syndromes, in combination with neostigmine.

Said 5HT3-antagonist is administered to said mammalian subject at a single dose of from 0.001 mg/kg to 1.8 mg/kg of body weight, given from one to three times per day, with a maximum of 300 mg/day, in combination with neostigmine.

For use in the treatment of muscle weakness associated with MG and other myasthenic syndromes in said combination with neostigmine, the 5HT3-antagonist is formulated in a pharmaceutical composition, wherein said 5HT3-antagonist is in admixture with a pharmaceutical carrier or vehicle.

In certain preferred embodiments, the present invention provides pharmaceutical compositions including, as one of their active ingredients, a pharmacologically active amount of a 5HT3-antagonist as shown above in “The 5HT3-antagonist” section or of one of its pharmaceutically acceptable salts, in mixture with a pharmaceutical carrier or vehicle.

In the pharmaceutical compositions of the present invention for oral, subcutaneous, intramuscular, intravenous, transdermal or topical administration, the 5HT3-antagonist active ingredient is preferably administered in the form of dosage units, in mixture with the classic pharmaceutical carriers or vehicles, in combination with neostigmine.

The posology can vary widely depending on the age, weight, and the health condition of the mammalian subject. This posology includes the administration of a dose of from 1 μg to 300 mg according to the potency of each 5HT3-antagonist and the age or weight of the mammalian subject, from one to three times a day by intramuscular, intravenous, subcutaneous, oral, or transcutaneous administration.

The pharmaceutical compositions of the present invention are formulated with the classic excipients suitable for different ways of administration. Particularly advantageous are the formulations in the form of tablets, multi-score tablets, coated tables, orally disintegrating tablets, extended release tablets, hard or soft capsules, extended-release capsules, patches for transdermal administration, liquid oral solutions, syrups or suspensions in a predetermined unit form, and vials for the intravenous or subcutaneous administration.

The aforementioned pharmaceutical composition comprising said 5HT3-antagonist, in the aforesaid amounts per unit form, is administered to a patient suffering from MG or another myasthenic syndrome in combination with neostigmine, also in a pharmaceutical composition in dosage unit form, comprising an effective amount of neostigmine in admixture with a pharmaceutical carrier. Said effective amounts, in unit forms for oral, intravenous, or subcutaneous for continuous infusion administration as well as the neostigmine daily doses, are illustrated in “The Neostigmine” section. Normally, said effective amount per unit form for oral administration is in the range of from 0.2 mg to 200 mg, preferably from 17.5 mg to 200 mg. Preferably, said neostigmine is neostigmine bromide. Normally, said effective amount per unit form for continuous subcutaneous infusion administration is from 10 mg to 500 mg, preferably from 60 mg to 240 mg. Preferably said neostigmine for continuous subcutaneous infusion is neostigmine methylsulfate.

Ondansetron and pharmaceutically acceptable salts and solvates thereof, dolasetron and pharmaceutically acceptable salts and solvates thereof, palonosetron and pharmaceutically acceptable salts and solvates thereof, ramosetron and pharmaceutically acceptable salts and solvates thereof, and tropisetron and pharmaceutically acceptable salts thereof are particularly preferred in said composition.

When the 5HT3-antagonist is ondansetron, the dose per tablet in combination with oral neostigmine will range from 0.5 mg to 32 mg, normally from 2 mg to 32 mg, from 2 mg to 16 mg, from 2 mg to 8 mg or from 4 mg to 8 mg. The dose per ampule for continuous 24-hour, subcutaneous infusion will be from 4 mg to 32 mg, or from 4 mg to 24 mg, preferably from 8 mg to 16 mg.

Ondansetron may also be present in a composition for transdermal administration, subcutaneous administration, intravenous administration, in a slow-release composition, such as extended release tablets or capsules, or in a combination product, for example in a Transdermal Drug Delivery System (TDDS) such as a patch, preferably a matrix patch like that described by Cho J-R et al 2016; a patch pump, an infusion pump, or a micropump; or a fast-dissolving buccal film such as that described by Koland M et al. 2013.

“Transdermal drug delivery system” provides transdermal delivery using transdermal drug formulations and transdermal patches incorporating such transdermal drug formulations. For example, the transdermal drug delivery system may include a composition in form of a patch, a cream, a gel, a lotion or a paste comprising a 5HT3-antagonist (such as ondansetron). Examples of transdermal formulations may include, but are not limited, to those as described in U.S. Pat. No. 6,562,368, a transdermal gel formulation as described in U.S. Pat. Nos. 7,029,694; 7,179,483; 8,241,662 and US 2009/0018190, a transdermal or transmucosal pharmaceutical formulation, that can be utilized for topical or transdermal application, such that solutions, creams, lotions, sprays, ointment, gels, aerosols and patch drug deliveries as described in WO 2005/039531, US2007/022379, US 2010/0216880, US 2014/0037713 and U.S. Pat. No. 8,652,491, a transdermal absorption preparation as described in WO2013/061969 and US 2014/0271796, the disclosures of which are herein incorporated by reference in their entirety. The transdermal patches may also include, but are not limited to, a patch pump having an in-dwelling rigid catheter with flexible features and/or a flexible catheter attachment as described in U.S. Pat. No. 9,782,536, a selectively activatable patch pump as described in U.S. Pat. No. 9,724,462, a patch pump attached to a wireless communication system as described in U.S. Pat. No. 9,623,173, a conformable patch pump as described in U.S. Pat. No. 9,616,171, an infusion pump as described in U.S. Pat. Nos. 8,915,879, 9,801,997, and 9,839,745, and 9,867,930, a portable infusion drug delivery as described in U.S. Pat. No. 8,480,649, a micropump as described in U.S. Pat. No. 8,282,366, and a patch pump as described in U.S. Pat. No. 7,828,771; the disclosures of which are herein incorporated by reference in their entirety. Other transdermal patches may include, but are not limited to, a patch in which oxybutynin is incorporated in an adhesive agent layer composition comprises the acrylic-based polymer as the adhesive base agent, and the acrylic-based polymer is a copolymer of polymethyl methacrylate with a polyacrylateas described in U.S. Pat. No. 8,802,134, a patch consisting of a support layer and of an adhesive agent layer arranged on the at least one surface of the support layer as described in U.S. Pat. No. 8,877,235, a patch using a monoglyceride or a mixture of monoglycerides of fatty acids as skin permeation-enhancer as described in U.S. Pat. Nos. 5,441,740 and 5,500,222, a patch for using a monoglyceride or a mixture of monoglycerides plus a lactate ester as skin permeation-enhancer as described in U.S. Pat. Nos. 5,686,097; 5,747,065; 5,750,137 and 5,900,250, a patch with a non-rate controlling tie layer on the skin-proximal surface of the reservoir, not affecting the drug release as described in U.S. Pat. Nos. 5,614,211 and 5,635,203, a patch using triacetin as permeation enhancer as described in U.S. Pat. Nos. 5,212,199, 5,227,169, 5,601,839 and 5,834,010, a patch with a matrix mass in the form of a layer which is self-adhesive, and in which the matrix mass consists of ammonium-group-containing (meth)acrylate copolymers as described in U.S. Pat. No. 6,555,129, a transdermal patch as described in U.S. Pat. Nos. 6,743,441; 7,081,249;7,081,250; 7,081,251; 7,081,252 and 7,087,241; the disclosures of which are herein incorporated by reference in their entirety. Preferably, the transdermal drug delivery system is a patch, a patch pump, an infusion pump, or a micropump.

When the 5-HT3 antagonist is dolasetron, the dose per tablet in combination with neostigmine will range from 100 mg to 200 mg of dolasetron.

When the 5HT3-antagonist is palonosetron hydrochloride in an IR-formulation, the dose per tablet to be used in combination with neostigmine is equivalent to 0.25 mg to 0.5 mg of palonosetron base. Said tablet is destined to be administered once a day or once every two days.

Preferably, said neostigmine is neostigmine bromide for oral administration or neostigmine methyl sulfate for parenteral administration.

In the treatment of symptoms of muscle weakness associated with MG and other myasthenic syndromes, the 5HT3-antagonist and the neostigmine are used in combination and the two active components may be co-administered simultaneously or sequentially, or in a fixed dose combination comprising of a pharmaceutical composition comprising the 5HT3-antagonist and neostigmine, in admixture with a pharmaceutically acceptable carrier or vehicle.

The 5HT3-antagonist Component (a) and the neostigmine Component (b) can be administered separately or together in any conventional oral or parenteral dosage unit form such as capsule, tablet, powder, sachet, suspension, solution, or transdermal device. The amount of 5HT3-antagonist per unit form in preferred embodiments will be in the range of from 1 μg to 300 mg. The amount of neostigmine per unit form in preferred embodiments will be in the range of from 1 mg to 200 mg.

In the case of separate (concurrent or sequential) administration of said 5HT3-antagonist, in an effective amount per unit form, and of said neostigmine, in an effective amount per unit form, each of them can be packaged in a kit comprising said 5HT3-antagonist, in admixture with a pharmaceutical carrier or vehicle, in a container; and said neostigmine, in admixture with a pharmaceutical carrier or vehicle, in another, separate container.

For the concurrent administration of said 5HT3-antagonist and of said neostigmine, the two active principles can be formulated together and with a pharmaceutical carrier or vehicle, in a pharmaceutical composition.

Accordingly, the present invention provides the use of a 5-HT3 antagonist for the preparation of a medicament for the treatment of symptoms of muscle weakness associated with MG and other myasthenic syndromes in combination with neostigmine, said medicament including a pharmaceutical composition in dosage unit form comprising said 5HT3-antagonist and said neostigmine pharmaceutically acceptable salt, in admixture with a pharmaceutical carrier or vehicle.

### Fourth Aspect of the Invention

According to a fourth aspect of the present invention, the pharmaceutical composition comprising a 5HT3-antagonist may contain another active ingredient, in particular neostigmine, co-formulated with said 5HT3-antagonist, in admixture with a pharmaceutical carrier or vehicle.

Thus, the present invention further provides a fixed-dose combination including a pharmaceutical or veterinary composition in dosage unit form comprising, as active ingredients,

Component (a): a 5HT3-antagonist; and
Component (b): neostigmine,
in admixture with a pharmaceutical carrier or vehicle.

Normally, in said composition, the 5HT3-antagonist Component (a) is present in an amount per unit form of from 1 μg to 300 mg and the neostigmine Component (b) for oral administration is present in an amount equivalent to from 0.2 mg to 200 mg, normally to from 17.5 mg to 200 mg of neostigmine bromide or for subcutaneous 24-hour continuous administration is present in an amount equivalent to 10 mg to 500 mg, or 30 mg to 400 mg, preferably 60 mg to 240 mg neostigmine methylsulfate.

Said fixed-dose combination is useful for the treatment of MG and other myasthenic disorders in a mammal such as a cat, a dog or a human being. Said treatment safely provides said mammal with a 5HT3-antagonist dose of from 1 μg to 300 mg and a single neostigmine dose equivalent to from 0.2 mg to 200 mg of neostigmine bromide or neostigmine methylsulfate.

When said mammal is a human being, the above fixed-dose combination may be safely used for the treatment of infants, including neonates, and also includes neostigmine doses for titration.

According to an embodiment,

said 5HT3-antagonist Component (a) active ingredient is selected from the group consisting of azasetron and pharmaceutically acceptable salts and solvates thereof, in an amount per unit form equivalent to from 5 mg to 10 mg of azasetron hydrochloride; dolasetron and pharmaceutically acceptable salts and solvates thereof, in an amount per unit form equivalent to from 25 mg to 200 mg of dolasetron mesylate; granisetron and pharmaceutically acceptable salts and solvates thereof, in an amount per unit form equivalent to from 0.5 mg to 2 mg of granisetron base; ondansetron and pharmaceutically acceptable salts and solvates thereof, in an amount per unit form equivalent to from 0.5 mg to 32 mg, from 2 mg to 32 mg, from 2 mg to 16 mg, or 2 mg to 8 mg of ondansetron base; palonosetron and pharmaceutically acceptable salts and solvates thereof, in an amount per unit form equivalent to from 0.25 mg to 0.5 mg of palonosetron base; and tropisetron and pharmaceutically acceptable salts and solvates thereof, in an amount per unit form equivalent to from 2.5 mg to 5 mg of tropisetron base; and

said neostigmine pharmaceutically acceptable salt Component (b) is in an amount per unit form equivalent to from 0.2 mg to 200 mg of neostigmine bromide or 10 mg to 240 mg neostigmine methylsulfate;

and the Components are mixed together and with a pharmaceutical carrier or vehicle.

According to a first aspect of this embodiment, the fixed-dose combination is a pharmaceutical composition in dosage unit form comprising

a 5HT3-antagonist Component (a) selected from the group consisting of azasetron hydrochloride, in an amount per unit form of from 5 mg to 10 mg; dolasetron mesylate, in an amount per unit form of from 25 mg to 200 mg; granisetron hydrochloride, in an amount per unit form equivalent to from 0.5 mg to 2 mg granisetron base; ondansetron hydrochloride dihydrate, in an amount per unit form equivalent to from 0.5 mg to 32 mg or from 2 mg to 16 mg of ondansetron base; palonosetron hydrochloride, in an amount per unit form equivalent to from 0.25 mg to 0.5 mg of palonosetron base; and tropisetron hydrochloride, in an amount per unit form equivalent to from 2.5 mg to 5 mg of tropisetron base; and

neostigmine bromide Component (b), in an amount per unit form of from 15 mg to 200 mg, from 17.5 mg to 200 mg, from 35 mg to 200 mg, from 50 mg to 200 mg, from 62.5 mg to 200 mg, from 70 mg to 200 mg and from 100 mg to 200 mg,

in admixture with a pharmaceutical carrier or vehicle in an oral formulation.

According to a second aspect of this embodiment, the fixed-dose combination is a pharmaceutical composition in dosage unit form comprising

a 5HT3-antagonist Component (a) selected from the group consisting of ondansetron hydrochloride dihydrate, in an amount per unit form equivalent to from 0.5 mg to 32 mg or from 2 mg to 16 mg of ondansetron base; and

neostigmine methylsulfate Component (b), in an amount per unit form of from 0.2 mg to 200 mg, from 17.5 mg to 200 mg, from 35 mg to 200 mg, from 50 mg to 200 mg, from 62.5 mg to 200 mg, from 70 mg to 200 mg and from 100 mg to 200 mg,

in an aqueous solution comprising a pharmaceutical carrier or vehicle in a parenteral formulation for injection or infusion.

In the above 5HT3-antagonist/neostigmine fixed dose combinations, the above-illustrated pharmaceutical compositions in dosage unit form are preferably administered to a pediatric or adult patient suffering from symptoms of muscle weakness associated with MG or another myasthenic syndrome to provide a neostigmine oral daily dose equivalent to from 1 mg to 1500 mg, and even more, normally from 15 mg to 1200 mg, from 17.5 mg to 1200 mg, from 270 mg to 1200 mg, from 375 mg to 1200 mg or from 450 mg to 1200 mg of neostigmine bromide or for continuous subcutaneous infusion from 10 mg to 500 mg, or 30 mg to 500 mg, or 60 mg to 240 mg of neostigmine methyl sulfate.

As set forth above, the pharmaceutical compositions are formulated in admixture with a pharmaceutical carrier or vehicle for any administration route. For example, said pharmaceutical compositions are in a pharmaceutical dosage unit form for oral, intravenous, intramuscular, intranasal, intraperitoneal, subcutaneous, transdermal, or rectal administration.

The pharmaceutical compositions may be formulated in oral forms such as tablets or gelatin capsules wherein the 5HT3-antagonist or neostigmine or both the active ingredients are in admixture with a carrier or vehicle that may include a diluent, such as cellulose, dextrose, lactose, mannitol, sorbitol or sucrose; a lubricant, such as, acid, calcium or magnesium stearate, polyethylene glycol, silica, or talc; and if needed, a binder such as magnesium aluminum silicate, gelatin, methylcellulose, sodium carboxymethylcellulose, or polyvinylpyrrolidone.

A typical oral pharmaceutical composition in IR-formulation may be a capsule comprising 35 mg of neostigmine bromide and an amount of ondansetron hydrochloride dihydrate equivalent to 6 mg of ondansetron base manufactured, for example, as described, for (R)-ondansetron alone, in U.S. Pat. No. 5,962,494, the disclosure of which is herein incorporated by reference in its entirety.

Said oral forms may be tablets coated with sucrose or with various polymers; or, alternatively, the tablets can be manufactured by using carriers such as acrylic and methacrylic acid polymers and copolymers; cellulose derivatives such as hydroxypropylethylcellulose; or other appropriate materials, to have a prolonged or delayed activity by progressively releasing a predetermined quantity of 5HT3-antagonist or of neostigmine, or of both the active ingredients. The oral formulations can also be in form of capsules allowing the extended release of the 5HT3-antagonist, or of neostigmine, or of both the active ingredients.

A typical oral tablet for oral administration comprising an amount of ondansetron hydrochloride dihydrate equivalent to 6 mg of ondansetron base and 35 mg of neostigmine bromide may be prepared according to conventional methods, for example as described, for (R)-ondansetron alone, in the aforementioned U.S. Pat. No. 5,962,494.

The unit forms may be formulated in tablets in which Component (a) or Component (b) or a mixture of the two components is in Extended Release (“ER”)-formulation, for example in admixture with hydroxypropyl methyl cellulose or in a film-coated microgranule. Carriers and vehicles for ER tablets include retardant materials such as acrylic and methacrylic acid polymers and copolymers; the aforementioned cellulose derivatives such as hydroxypropylmethylcellulose, hydroxyethyl cellulose, hydroxypropylethylcellulose, hydroxypropylcellulose, methylcellulose, ethylcellulose, or sodium carboxymethylcellulose; gums; waxes; glycerides or aliphatic alcohols or a mixture thereof

When the 5HT3-antagonist and neostigmine are in a fixed-dose combination, the unit form may be a stratified, bi-layer tablet wherein the 5HT3-antagonist, formulated with a pharmaceutical carrier, is in one of the layers and neostigmine, formulated with a pharmaceutical carrier, is the other layer. Similarly, the 5HT3-antagonist and neostigmine active ingredients are in a pill containing one of the active ingredients, admixed with a pharmaceutical carrier, in the core and the other active ingredient, admixed with a pharmaceutical carrier, is in the outer part of the pill, the core and the outer part being optionally separated by an inert film or carrier. Analogously, capsules made of two separated parts, one containing Component (a), in IR- or ER-formulation and the other containing Component (b), in IR- or ER-formulation, may be manufactured.

The fixed-dose combinations may also be pharmaceutical compositions formulated as an orally disintegrable tablet wherein Component (a) and Component (b) are mixed together and with a hydrophobic agent and a diluent to form a fast release composition which efficiently delivers said components orally, for example as disclosed, for ondansetron Component (a) alone, in GB 1548022, GB 2111423, GB 2119246, GB 2114440, GB 2111184, GB 2120370, and U.S. Pat. Nos. 5,046,618 5,188,825, 5,955,488, 7,390,503 and in WO 2004/096214, the disclosures of which are incorporated herein in their entirety by reference and for neostigmine Component (b) alone, in WO 2006/005017, the disclosure of which is incorporated herein in its entirety by reference.

An useful pharmaceutical composition according to the present invention is formulated in a liquid formulation, such, as a syrup, wherein Component (a) and Component (b) are dissolved in admixture with a pharmaceutical carrier, for example, for ondansetron Component (a) alone, as described in U.S. Pat. No. 5,854,270 the disclosure of which is incorporated herein in its entirety by reference.

Said compositions in form of orally disintegrable tablets or syrups may also comprise sweeteners, lubricants, taste-masking agents, binders, coloring agents and, in the case of orally disintegrable tablets, salivation stimulants.

A typically orally disintegrable tablet will contain an amount of ondansetron hydrochloride dihydrate equivalent to from 0.5 mg to 32 mg or from 2 mg to 16 mg of ondansetron base, normally an amount of ondansetron hydrochloride dihydrate equivalent to 0.5 mg, 2 mg, 4 mg, 6 mg, 8 mg or 16 mg of ondansetron base, as Component (a); and 15 mg, 17.5 mg, 35 mg, 50 mg, 62.5 mg, 70 mg, 100 mg or 200 mg of neostigmine bromide, as Component (b).

A typical syrup will contain an amount of from 2 mg/5 ml to 8 mg/5 ml of ondansetron base or an amount of ondansetron hydrochloride dihydrate Component (a) equivalent to from 2 mg/ml to 8 mg/ml of ondansetron base; and from 30 mg/5 ml to 60 mg/5 ml of neostigmine bromide Component (b).

A syrup, preferably destined to a pediatric patient, to a cat or to a dog, will comprise an amount of ondansetron hydrochloride dihydrate Component (a) equivalent to from 0.5 mg/ml to 2 mg/ml of ondansetron base and an amount of from 2 mg/ml to 15 mg/ml of neostigmine bromide.

The pharmaceutical compositions may also be formulated in a transdermal drug delivery system (TDDS), such as a patch formulation wherein the active ingredient or the mixture of the active ingredients may comprise adjuvants such as D-sorbitol, gelatin, kaolin, methyl paraben, polysorbate 80, propylene glycol, propyl paraben, povidone, sodium carboxymethylcellulose, sodium polyacrylate, tartaric acid, titanium dioxide, and purified water. A patch formulation may also contain skin permeability enhancer such as lactate esters such as lauryl lactate, triacetin or diethylene glycol monoethyl ether.

In embodiments of the above pharmaceutical compositions, a preferred 5HT3-antagonist Component (a) active ingredient is selected from the group consisting of ondansetron base, ondansetron hydrochloride dihydrate, palonosetron base, palonosetron hydrochloride, dolasetron base, and dolasetron mesylate monohydrate; and the preferred pharmaceutically acceptable salt of neostigmine is neostigmine bromide. Each of these active ingredients is present in said compositions in the amount per unit form illustrated herein above.

According to an embodiment, the compositions of the present invention are formulated by mixing Component (a) and Component (b) together, in admixture with a pharmaceutical carrier for an immediate release. An advantageous composition according to this embodiment comprises ondansetron hydrochloride dihydrate, in an amount equivalent to from 0.5 mg to 32 mg or from 2 mg to 24 mg of ondansetron base, as Component (a); and from 0.2 mg to 200 mg of neostigmine bromide, as component (b). Components (a) and (b) are mixed together and with a pharmaceutical carrier in an IR- or ER-formulation. Said composition is destined to be administered from two to seven times per day.

Another composition in dosage unit form according to this embodiment comprises ondansetron hydrochloride dihydrate, in an amount equivalent to from 0.5 mg to 2 mg ondansetron base, as Component (a); and from 35 mg to 100 mg, normally from 17.5 mg to 100 mg, of neostigmine bromide, as Component (b). Components (a) and (b) are mixed together and formulated with a pharmaceutical carrier in an IR-coated tablet. Two coated tablets comprising this composition may be administered from two to seven times per day to mammalian subjects, and particularly, humans, dogs, and cats, suffering from conditions or symptoms of muscle weakness associated with MG or other myasthenic syndromes. Such a treatment safely improves said conditions or symptoms.

### EXAMPLE 1

The ability of the 5HT3-antagonists for preventing the gastro-intestinal adverse effects of orally administered neostigmine bromide in humans was tested.

A Phase I study was conducted in six human subjects receiving a single oral dose of neostigmine bromide with or without a single oral dose of ondansetron hydrochloride dihydrate, as a representative 5HT3-antagonist. The study was a single center, single-blind.

The objective of the study was to demonstrate that ondansetron could safely attenuate the gastro-intestinal side effects of neostigmine given in doses demonstrated to be effective for the treatment of Myasthenia Gravis.

To be enrolled in the study, participants (aged 18 to 60 years of age) were required, to refrain from consuming xanthine, quinine and caffeine containing beverages, and to refrain from prolonged intensive physical exercise during the study conduct. All subjects signed an informed consent form indicating that they understood the purpose of and procedures required for the study and that they were willing to participate in the study and comply with the study procedures and restrictions. The key criteria for exclusion of a subject from enrollment in the study were as follows:

any clinically relevant acute or chronic disease which could interfere with the subjects' safety during the trial, expose them to undue risk, or interfere with the study objectives;
history or presence of gastrointestinal, hepatic, or renal disease or other condition known to interfere with the absorption, distribution, metabolism or excretion of drugs;
history of substance abuse, known drug addiction, or positive test for drugs of abuse or alcohol;
history of drug or other significant allergy;
ECG changes including QT interval prolongation and congenital long QT syndrome. Electrolyte abnormalities (e.g., hypokalemia or hypomagnesemia), congestive heart failure, bradyarrhythmias or other conditions that lead to QT prolongation;
treatment with centrally active drugs or those affecting peripheral cholinergic transmission within 3 months of study entry;
smokers (except subjects who stopped smoking 1 year or more before enrollment in the Study);
excessive daily consumption of xanthines containing drinks (i.e. >500 mg/day of caffeine);
intake of an investigational drug within 30 days of study entry.

Following enrollment in the study, participants received single increasing oral doses of neostigmine, given once daily in the morning. Once a subject had reached his/her first intolerable dose, upward dose escalation was discontinued. First intolerable dose was defined as

(a) one episode of vomiting; or
(b) two episodes of retching; or
(c) one episode of severe nausea; or
(d) one episode of moderate diarrhea (Grade 2).

Following a wash-out, participants then received their first intolerable dose of neostigmine plus a single oral dose of oral ondansetron hydrochloride dihydrate (10 mg, equivalent to 8 mg ondansetron base). If this first intolerable dose of neostigmine taken with ondansetron was tolerated, the dose of neostigmine was further increased together with oral ondansetron hydrochloride dihydrate (10 mg, equivalent to 8 mg ondansetron base) until subjects again reached an intolerable dose (FID-2).

On each study day, subjects were followed up for up to 8 hours for AEs and vital signs. A laboratory panel was taken at screening and at the end of the study.

Results showed that the co-administration of ondansetron with neostigmine attenuated gastro-intestinal AEs reported with neostigmine alone, and enabled subjects to reach neostigmine doses as high as or higher than the recommended efficacious dose for the treatment of MG prior to these subjects reaching FID-2.

In conclusion, the co-administration of oral high dose ondansetron with neostigmine prevented the occurrence of gastro-intestinal AEs given in doses as high as or higher than the recommended efficacious dose for the treatment of myasthenia gravis.

The foregoing detailed description has been given for illustration purposes only, especially for purposes of clarity of understanding. It will be apparent to those skilled in the art that certain changes and modifications may be practiced without departing from the spirit and scope of the invention, which is delineated by the appended claims.

### REFERENCES

Abicht A, Muller J S, Lochmülller H. Congenital Myasthenic Syndromes. In: Pagon R A, Adam M P, Ardinger H H, Wallace S E, Amemiya A, Bean L J H, Bird T D, Ledbetter N, Mefford H C, Smith R J H, Stephens K, editors. GeneReviews® [Internet]. Seattle (Wash.): University of Wash., Seattle; 1993-2016. 2003 May 9 [updated 2016 Jul. 14].
   Cho J-R et al 2016: Cho J-R, Duong A V, Nguyen L T T, Chi S-C. “Design of transdermal matrix patch containing ondansetron”. J Pharm Investigation. 2016 46(7): 677-684.
   Drachman D B. Myasthenia Gravis. Semin Neurol. 2016; 36:419-424. Epub 2016 Sep. 23.
   Engel AG. Congenital Myasthenic Syndromes in 2012. Curr. Neurol Neurosci Rep, 2012; 12:92-101.
   Gotterer L, Li Y. Maintenance immunosuppression in myasthenia gravis. J Neurol Sci. 2016; 369:294-302. Epub 2016 Aug. 28.

### Howard J. F. Clinical Overview of MG. Myastenia Gravis Foundation of America; 2015.

Koland M et al. 2010: Koland M, Sandeep V P. Charyulu N R. “Ondansetron Hydrochloride: Effect of Additives on in vitro Drug Release and Mucosal Permeation”. J Young Pharmacists. 2010, 2(3):216-222.
O'Grady GL, Verschuuren C, Yuen M, Webster R, Menezes M, Fock J M, Pride N, Best H A, Benavides Damm T, Turner C, Lek M, Engel A G, North K N, Clarke N F, MacArthur D G, Kamsteeg E J, Cooper S T. Variants in SLC18A3, vesicular acetylcholine transporter, cause congenital myasthenic syndrome. Neurology. 2016; 87:1442-1448. Epub 2016 Sep. 2.
Phillips W D1, Vincent A2. Pathogenesis of myasthenia gravis: update on disease types, models, and mechanisms. F1000Res. 2016; 27:5.
Shelton G D1. Myasthenia gravis and congenital myasthenic syndromes in dogs and cats: A history and mini-review. Neuromuscul Disord. 2016; 26: 331-334. Epub 2016 Mar. 10.
Smith S V, Lee A G. Update on Ocular Myasthenia Gravis. Neurol Clin. 2017; 35:115-123.

1. A method for safely improving the conditions of a mammal suffering from symptoms of muscle weakness associated with MG or another myasthenic syndrome, comprising chronically administering to said mammal a 5HT3-antagonist in combination with neostigmine.

2. The method of claim 1, wherein said neostigmine is neostigmine bromide or neostigmine methyl sulfate.

3. The method of claim 1, wherein said 5HT3-antagonist is administered to said mammal at a unit oral or subcutaneous dose of from 0.001 mg/kg to 1.8 mg/kg, given from one to three times per day, with a maximum of 300 mg/day.

4. The method of claim 1, wherein said 5HT3-antagonist is ondansetron or a pharmaceutically acceptable salt or solvate thereof.

5. The method of claim 4, wherein said ondansetron or pharmaceutically acceptable salt or solvate thereof is administered at a unit dose equivalent to from 0.5 mg to 32 mg of ondansetron base.

6. The method of claim 4, wherein said ondansetron or pharmaceutically acceptable salt or solvate thereof is ondansetron hydrochloride dihydrate, administered by continuous infusion at an unit dose equivalent to from 0.021 mg/h to 1 mg/h of ondansetron base.

7. The method of claim 6, wherein said ondansetron or pharmaceutically acceptable salt or solvate thereof is ondansetron hydrochloride dihydrate, administered by continuous infusion at a dose equivalent to from 0.5 mg/h to 1 mg/h of ondansetron base.

8. The method of claim 1, wherein said neostigmine in said combination is administered to said mammal at a unit dose equivalent to from 0.03 mg/kg to 6.25 mg/kg of neostigmine bromide or neostigmine methyl sulfate.

9. The method of claim 1, wherein said neostigmine in said combination is administered to said mammal in a dosage unit form comprising an amount per unit form of said neostigmine equivalent to from 0.2 mg to 200 mg of neostigmine bromide or neostigmine methylsulfate.

10. The method of claim 1, wherein said neostigmine in said combination is administered to said mammal by a continuous 24 h-infusion subcutaneous dose equivalent to from 0.09 mg/24 h to 500 mg/24 h of neostigmine methyl sulfate.

11. The method of claim 10, wherein said continuous 24 h-infusion subcutaneous dose is equivalent to from 10 mg/24 h to 500 mg/24 h of neostigmine methylsulfate.

12. The method of claim 1, wherein said neostigmine in said combination is administered to said mammal at a daily dose equivalent to from 0.2 mg to 1500 mg of neostigmine bromide or neostigmine methyl sulfate.

13. The method according to claim 1, wherein the mammal is suffering from myasthenia gravis.

14. The method according to claim 1, wherein the mammal is a human, dog or cat.

15. The method according to claim 14, wherein the mammal is a human.

16. A method for treating or alleviating symptoms of muscle weakness associated with a myasthenic syndrome, comprising administering to a mammalian subject in need thereof, an effective daily dose of a combination comprising 5HT3-antagonist and neostigmine.

17. A fixed-dose combination comprising a pharmaceutical or veterinary composition in dosage unit form comprising, as active ingredients,
Component (a) a 5HT3-antagonist; and
Component (b) neostigmine,
in admixture with a pharmaceutical carrier or vehicle.

18. The fixed-dose combination of claim 17, wherein the 5HT3-antagonist Component (a) is present in an amount per unit form of from 1 μg to 300 mg and the neostigmine Component (b) is present in an amount equivalent to from 0.2 mg to 500 mg of neostigmine bromide or methylsulfate.
